(19) United States
(12) Patent Application Publication
(10) Pub. No.: US 2004/0230136 A1
(43) Pub. Date: Nov. 18, 2004

(54) GUIDEWIRE HAVING AXIALLY EXTENDING FLOW THROUGH PASSAGEWAYS

(76) Inventor: Richard F. Corrigan JR., Wheaton, IL (US)

Correspondence Address: HOVEY WILLIAMS LLP, 2405 GRAND BLVD., SUITE 400, KANSAS CITY, MO, 64108, US

(21) Appl. No.: 10/249,880

(22) Filed: May 14, 2003

(51) Int. Cl. A61B 5/00

(52) U.S. Cl. 600/585

(57) ABSTRACT

A guidewire insertable into a body lumen and presenting a plurality of longitudinally extending ridges.

### BACKGROUND OF INVENTION

1. Field of the Invention

This invention relates generally to guidewires which can be inserted into and advanced through body lumens. In another aspect, the present invention relates to guidewires having shape memory which allow the wires to be steered through blood vessels to desired body locations.

2. Discussion of Prior Art

Conventional guidewires used for angioplasty, stent delivery, atherectomy, and other vascular procedures usually comprise an elongated flexible wire core member and a highly flexible atraumatic tip (e.g., a helical coil or tubular body of polymeric material) coupled to the distal end of the wire core. A torquing means is provided on the proximal end of the wire core to rotate, and thereby steer the guidewire while it is being advanced through a patient's vascular system.

The “steerability” of a guidewire (i.e., its ability to react to torquing and pushing forces applied by the user so that the distal end may be manipulated into passageways of interest) is primarily determined by the torsional stiffness and column strength of the wire core. When the user rotates the wire core about its longitudinal axis at the wire core's proximal end, the distal end should rotate through substantially the same angle so that the user can sense what is happening to the distal end as he or she rotates the proximal end. Ideally, there should be a one-to-one correspondence between the torque angle applied by the user at the proximal end and the angular rotation of the distal end. To approach this ideal, it is recognized that the wire core should exhibit an exceptionally high torsional stiffness. On the other hand, the wire core's column strength must be sufficient to provide the wire core with enough stiffness to enable it to be pushed through lesions, other obstructions, or tourchous vessels yet provide the wire with sufficient flexibility as to enable the wire to follow an arterial passageway without straightening the vessel, causing trauma to the vessel, or worse yet, perforating the vessel. Thus, it will be appreciated that the ideal column strength of a wire core is a trade-off between these competing traits.

In order to provide the desired steerability of a guidewire, the wire core is typically composed of a material exhibiting a high resistance to permanent distortion upon being bent (i.e., a good shape memory). One such material exhibiting good shape memory is a nickel-titanium alloy (available from Nitinol Devices & Components, Freemont, Calif.). While nickel-titanium alloys are advantageous because of their good shape memory, they are disadvantageous from the standpoints of its cost, difficulty to process, and tendency of being too flexible.

In a typical coronary procedure using a guidewire, a guiding catheter having a preformed distal tip is percutaneously introduced into a patient's peripheral artery (e.g., femoral or brachial artery) by means of a conventional Seldinger technique and advanced and steered therein until the distal tip of the guiding catheter is seated in the ostium of a desired coronary artery. There are two basic techniques for advancing a guidewire into the desired location within the patient's coronary anatomy through the in-place guiding catheter. The first is a preload technique which is used primarily for over-the-wire (OTW) devices and the second is the bare wire technique which is used primarily for rail-type systems.

With the preload technique, a guidewire is positioned within an inner lumen of an OTW device (e.g., a dilatation catheter or stent delivery catheter) with the distal tip of the guidewire just proximal to the distal tip of the catheter and then both are advanced through the guiding catheter to the distal end thereof. The guidewire is then advanced out of the distal end of the guiding catheter and into the patient's coronary vasculature until the distal end of the guidewire crosses the arterial location where the interventional procedure is to be performed (e.g., a lesion to be dilated or a dilated region where a stent is to be deployed). The catheter, which is slidably mounted onto the guidewire, is then advanced out of the guiding catheter into the patient's coronary anatomy over the previously introduced guidewire until the operative portion of the intravascular device (e.g., the balloon of a dilatation or stent delivery catheter) is properly positioned across the arterial location. Once the catheter is in position with the operative means located within the desired arterial location, the interventional procedure is performed. The catheter can then be removed from the patient over the guidewire.

With the bare wire technique, the guidewire is first advanced by itself through the guiding catheter until the distal tip of the guidewire extends beyond the arterial location where the procedure is to be performed. Then a rail-type catheter is mounted onto the proximal portion of the guidewire which extends out of the proximal end of the guiding catheter located outside of the patient. The rail-type catheter is then advanced over the guidewire, while the position of the guidewire is fixed, until the operative means on the rail-type catheter is disposed within the arterial location where the procedure is to be performed. After the procedure, the intravascular device may be withdrawn from the patient over the guidewire or the guidewire advanced further within the coronary anatomy for an additional procedure.

The wire core portion of a conventional guidewire has a generally circular cross-section. As such, a substantial portion of the outer surface of the wire core remains in contact with the medical device being slid over the wire core or the inner wall of the body lumen within which the wire core is received. Such a high area of surface contact can cause increased friction between the outer surface of the guidewire and the medical device (e.g., rail-type catheter) to be slid over the guidewire. Further, the generally circular cross-sectional area of the wire core can increase frictional resistance to sliding of the guidewire through the body lumen. In addition, the generally circular cross-section of conventional wire cores can prevent the injection of fluids, such as medications or a contrast medium, past the wire core.

### SUMMARY OF INVENTION

In view of the foregoing, it is an object of the present invention to provide a guidewire having optimal torsional stiffness, column strength, and shape memory while minimizing the amount of material required to form the wire core.

Another object of this invention is to provide a guidewire which reduces friction between the outer surface of the wire core and a body lumen wall, as well as between the outer surface of the wire core and a medical device to be slid over the wire.

A further object of this invention is to provide a guidewire having longitudinally extending passageways which allow for fluids to be injected past the guidewire and through a conduit (e.g., a rail-type catheter) within which the guidewire is positioned.

Still another object of this invention is to provide a guidewire having an increased outer surface area which, when exposed to heat treatment, provides for improved shape memory, axial stiffness, and torsional stiffness of the wire core.

It should be noted that the above-listed objects and advantages need not all be accomplished by the invention claimed herein and further aspects and advantages of the invention will be apparent from the following description of the invention and the appended claims.

In one aspect of the present invention, a guidewire insertable into a body lumen is provided. The guidewire comprises a monolithic wire presenting a plurality of longitudinally extending ridges presenting generally smooth outer surfaces.

In accordance with another aspect of the present invention, a guidewire insertable into a body lumen is provided. The guidewire comprises a solid wire having an outer surface which defines a plurality of primarily longitudinally extending grooves.

### BRIEF DESCRIPTION OF DRAWINGS

Preferred embodiments of the invention are described in detail below with reference to the attached drawing figures, wherein:

FIG. 1 is a plan view of a guidewire received in a coiled storage sheath;

FIG. 2 is a partial sectional side view of a guidewire constructed in accordance with the principles of the present invention; and

FIG. 3 is a sectional view of the wire core taken along line 3-3 in FIG. 2, particularly illustrating the shape of the outer surface of the wire core.

### DETAILED DESCRIPTION

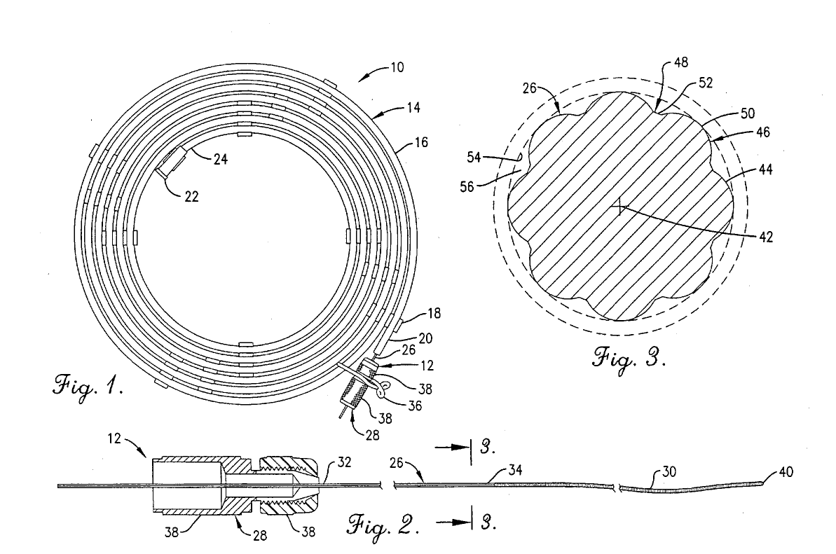

Referring initially to FIG. 1, a guidewire system 10 constructed in accordance with the principles of the present invention is illustrated as generally comprising a guidewire 12 and a coiled sheath 14. Coiled sheath 14 includes an elongated tube 16 which is wound into a generally flat, coiled configuration and held in such coiled configuration by a plurality of fasteners 18. Tube 16 has a generally annular cylindrical cross-section which provides for a longitudinally-extending internal passageway within which at least a portion of guidewire 12 can be received. An outer end 20 of tube 16 presents an opening through which at least a portion of guidewire 12 can be inserted and retracted. An end piece 22 is coupled to an inner end 24 of tube 16 and provides an opening through which saline can be injected into tube 16 for lubrication of guidewire 12 prior to insertion into the body. Coiled sheath 14 is configured to prevent damage to guidewire 12 during shipping and storage of guidewire 12.

Referring now to FIGS. 1 and 2, guidewire 12 generally comprises a flexible wire core 26, a torquing element 28, and a highly flexible atraumatic tip 30. Torquing element 28 is fixedly coupled to a proximal end 32 of wire core 26. Atraumatic tip 30 is fixedly coupled to a distal end 34 of wire core 26. When guidewire 12 is received in coiled sheath 14, as shown in FIG. 1, both wire core 26 and tip 30 are received in sheath 14, while torquing element 28 is positioned proximate outer end 20 of tube 16 and is held in position relative to tube 16 by a clip 36.

Referring to FIG. 2, torquing element 28 is preferably a generally cylindrical member having an outer diameter which allows for guidewire 12 to be readily rotated when torquing element 28 is manipulated by the hand of an operator. Preferably, at least a portion of the outer surface of torquing element 28 is comprised of a gripping surface 38 which facilitates manipulation of torquing element 28 by the operator. Atraumatic tip 30 can be a helical coil which is wound around distal end 34 of wire core 26. A rounded tip 40 is coupled to the distal end of atraumatic tip 30 in order to allow guidewire 12 to be inserted through a body lumen without piercing the wall of the body lumen.

Referring to FIGS. 2 and 3, wire core 26 is preferably an elongated, solid member which defines a longitudinal axis 42 extending along its geometric center. As used herein, the term “solid” shall mean having substantially no internal holes or openings. Preferably, wire core 26 is integrally formed. As used herein, the term “integrally formed” shall mean formed without physically binding two or more separately formed wire filaments to one another.

Referring to FIG. 3, wire core 26 presents an outer surface 44 having a generally non-circular cross-section. As used herein, the term “cross-section” shall mean a section taken perpendicular to the direction of extension of longitudinal axis 42. Outer surface 44 is preferably at least partly defined by a plurality of primarily longitudinally extending ridges 46 and a plurality of primarily longitudinally extending grooves 48. As used herein, the term “primarily longitudinally extending” shall mean extending in a direction which is skewed less than 45 degrees relative to the direction of extension of the longitudinal axis. Each groove 48 is positioned between a respective pair of ridges 46. Ridges 46 and grooves 48 are preferably at least substantially symmetrically spaced on outer surface 44 around longitudinal axis 42. Outer surface 44 of wire core 26 preferably includes from 3 to 16 ridges 46, more preferably from 4 to 12 ridges 46, and most preferably 5 to 8 ridges 46.

Each ridge 46 includes a crest 50, while each groove 48 includes a trough 52. Crest 50 is located on ridge 46 at a position where the radial distance between longitudinal axis 42 and outer surface 44, measured perpendicular to longitudinal axis 42, is maximum. Trough 52 is located in groove 48 at a position where the radial distance between longitudinal axis 42 and outer surface 44, measured perpendicular to longitudinal axis 42, is minimum. The average radial distance between longitudinal axis 42 and crests 50 is substantially greater than the average radial distance between longitudinal axis 42 and troughs 52. As used herein, the term “average radial distance” shall mean the sum of all the radial distances between longitudinal axis 42 and each crest 50 or trough 52, measured per perpendicular to longitudinal axis 42, divided by the total number of crests 50 or troughs 52, respectively. Preferably, the average radial distance between longitudinal axis 42 and crests 46, should be from about 2 percent to about 80 percent greater than the average radial distance from longitudinal axis 42 to troughs 52. More preferably, the average radial distance between longitudinal axis 42 and crests 50 is from about 5 percent to about 50 percent greater than the average radial distance from longitudinal axis 42 to troughs 52. Most preferably, the average radial distance from longitudinal axis 42 to crests 50 is from 8 percent to 25 percent greater than the average radial distance from longitudinal axis 42 to troughs 52.

Wire core 26 is adapted to be slidably received within a body lumen. Wire core 26 is also adapted to allow medical devices (e.g., rail-type or over-the-wire-type catheters) to be slid over outer surface 44 of wire core 26. It is, therefore, desirable for the frictional resistance between outer surface 44 of wire core 26 and an inner surface 54 of a body lumen wall or medical device to be minimized. Thus, it is preferred for ridges 46 and grooves 48 to extend in a direction which is substantially parallel to the direction of extension of longitudinal axis 42. Ridges 46 and grooves 48 preferably extend at least substantially continuously between proximal end 32 and distal end 34 of wire core 26. Further, it is preferred that the portions of outer surface 44 defined by ridges 46 present a generally smooth, most preferably arcuate, cross-section. The generally non-circular outer surface 44 of wire core 26 allows less surface area of wire core 26 to be contacted with inner surface 54, thereby reducing the frictional forces therebetween.

The shape of outer surface 44 of wire core 26 allows longitudinal passageways 56 to be formed between outer surface 44 and inner surface 54. Passageways 56 can allow a lubricating fluid to be injected therethrough and/or stored therein. When outer surface 44 is rotated relative to inner surface 54 the injected/stored lubricant in passageways 56 can lubricate the interface between outer surface 44 and inner surface 54, thereby reducing friction therebetween. Passageways 56 can also provide a conduit through which a fluid, such as a contrast medium, can be injected when wire core 26 is disposed in a body lumen.

The generally non-circular cross section of outer surface 44 provides wire core 26 with a larger surface area than if outer surface 44 were circular in shape. One advantage of the increased surface area of wire core 26 is realized when the material forming wire core 26 is heat treated. Wire core 26 is preferably formed of a material which has a superior shape memory. Preferably, wire core 26 is formed of a material selected from the group consisting of stainless steel, a nickel-titanium alloy, and a nickel-titanium-cobalt alloy. Most preferably, wire core 26 is formed of a nickel-titanium-cobalt alloy having the formula TiNiₓCoₗ₋ₓ wherein Ti denotes titanium and constitutes approximately 50 atomic percent of the composition, and the term NiₓCoₗ₋ₓ denotes nickel and cobalt respectively and make up the remaining approximately 50 atomic percent of the composition. X is a factor which varies from greater than zero to less than 1 whereby the relative percentage of nickel and cobalt varies inversely from less than 100 percent to more than zero percent. The transitional temperature of this alloy can be varied depending upon relative composition from −396° to 331° F. One example of a suitable nickel-titanium-cobalt alloy is commercially known as Nitinol™ and is available from Nitinol Devices & Components, Freemont, Calif.

Typically, when such metals or metal alloys, as described above, are used to form wire core 26, those metals or metal alloys require heat-treatment in order to exhibit sufficient torsional stiffness, column strength, and shape memory.

Conventional heat-treatment of such metals and metal alloys typically only affects the outer surface of wire core 26 and a small inner portion of wire core 26 immediately adjacent outer surface 44 (i.e., the depth of the heat-treatment is minimal). Thus, by increasing the surface area of wire core 26, more of wire core 26 is exposed to heat treatment, thereby providing for increased torsional stiffness, column strength, and shape memory of wire core 26.

Wire core 26 generally has a length, measured between proximal end 32 and distal end 34 of from about 20 to about 1000 centimeters, more preferably from about 50 to about 750 centimeters, and most preferably from 80 to 500 centimeters. The maximum diameter of wire core 26 is preferably in the range of from about 0.005 inches to about 0.10 inches, more preferably from about 0.01 inches to about 0.05 inches, and most preferably from 0.02 to 0.03 inches. As used herein, the term “maximum diameter” shall mean the maximum distance between any two points on outer surface 44 of the wire core 26, measured by a straight line extending through and perpendicular to longitudinal axis 42.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventor hereby states his intent to rely on the Doctrine of Equivalents to determine and assess the reasonably fair scope of the present invention as pertains to any apparatus not materially departing from but outside the literal scope of the invention as set forth in the following claims.

1. A guidewire insertable into a body lumen, said guidewire comprising:
a monolithic wire core presenting a plurality of longitudinally extending ridges,
each of said ridges having a generally smooth outer surface.

2. A guidewire according to claim 1,
said wire core being solid.

3. A guidewire according to claim 1,
each of said ridges presenting an outer surface having a generally arcuate cross-section.

4. A guidewire according to claim 1,
said wire core defining a longitudinal axis extending longitudinally along the geometric center of the wire core,
said ridges extending substantially parallel to the longitudinal axis.

5. A guidewire according to claim 4,
said ridges being generally symmetrically spaced around the longitudinal axis.

6. A guidewire according to claim 5,
said wire core presenting a plurality of longitudinally extending grooves,
each of said grooves positioned between a respective pair of ridges.

7. A guidewire according to claim 6,
each of said ridges including a crest representing the portion of the ridge spaced farthest from the longitudinal axis,
each of said grooves including a trough representing the portion of the groove spaced closest to the longitudinal axis,
the average radial distance from the longitudinal axis to the crests, being from about 2 percent to about 80 percent greater than the average radial distance from the longitudinal axis to the troughs.

8. A guidewire according to claim 7,
said wire core comprising from 3 to 16 ridges.

9. A guidewire according to claim 7,
said wire core comprising from 4 to 12 ridges.

10. A guidewire according to claim 8,
said wire core having a maximum diameter of from about 0.005 inches to about 0.1 inches.

11. A guidewire according to claim 10,
the average radial distance from the longitudinal axis to the crests, being from about 5 to about 50 percent greater than the average radial distance from the longitudinal axis to the troughs.

12. A guidewire according to claim 5,
said wire core being integrally formed.

13. A guidewire insertable into a body lumen, said guidewire comprising:
a solid, wire core having an outer surface which presents a plurality of primarily longitudinally extending grooves.

14. A guidewire according to claim 13,
said grooves being substantially symmetrically spaced around the outer surface of the wire.

15. A guidewire according to claim 14,
said wire core defining a longitudinal axis extending longitudinally along the geometric center of the wire,
said grooves extending substantially parallel to the longitudinal axis.

16. A guidewire according to claim 15,
said wire core presenting a plurality of longitudinally extending ridges,
each of said ridges positioned between a respective pair of grooves,
each of said ridges presenting a generally smooth outer surface.

17. A guidewire according to claim 16,
each of said ridges having a generally arcuate cross-section.

18. A guidewire according to claim 16,
said wire core comprising from 3 to 16 ridges.

19. A guidewire according to claim 16,
said wire core comprising from 4 to 12 ridges.

20. A guidewire according to claim 18,
said wire core being integrally formed.
